(19) United States
(12) Patent Application Publication
(10) Pub. No.: US 2006/0240120 A1
(43) Pub. Date: Oct. 26, 2006

(54) Composition for lowering blood glucose

(75) Inventor: Byoung-Rae Lee, Gwangju (KR)

Correspondence Address: ROTHWELL, FIGG, ERNST & MANBECK, P.C., 1425 K STREET, N.W., SUITE 800, WASHINGTON, DC, 20005, US

(73) Assignee: Hyundeok Bio & Technology Co., Ltd., Seoul (KR)

(21) Appl. No.: 10/562,481

(22) PCT Filed: Jun. 24, 2004
(86) PCT No.: PCT/KR04/01523

§ 371 Date: Jun. 5, 2006

(30) Foreign Application Priority Data
Jun. 25, 2003 (KR) 10-2003-1649

(51) Int. Cl. A61K 36/82; A61K 33/42; A61K 33/06; A61K 31/19

(52) U.S. Cl. 424/603; 424/682; 424/729; 514/557

(57) ABSTRACT

The present invention relates to a composition for lowering blood glucose, which contains a polyphenol extracted, isolated, and purified from green tea, and calcium, as active ingredients. The inventive composition shows a synergistic blood glucose-lowering effect by the green tea polyphenol and the calcium, and thus will be useful for diabetic patients.

### BACKGROUND OF THE INVENTION

1. Technical Field

The present invention relates to a composition for lowering blood glucose, which contains a polyphenol extracted, isolated, and purified from green tea, and calcium.

2. Background Art

Diabetes is a condition where blood glucose increases, and the most important factor in diabetic therapy is to lower blood glucose to a normal range. Blood glucose may be lowered by a reduction in carbohydrate intake or an increase in carbohydrate consumption. The staple of the Korean diet is boiled rice so that the intake of starch among carbohydrates is particularly high. Starch is decomposed into disaccharides or trisaccharides by saliva, starch digestive enzyme, or amylase contained in pancreatic juice, and they are decomposed into monosaccharide glucose by α-glycosidase in the small intestines and absorbed, resulting in an increase in blood glucose.

Acarbose™ which is an agent for inhibiting α-glycosidase, disaccharidase, in the starch digestive process, is now marketed as a diabetes therapeutic agent with the function of lowering blood glucose. However, since the α-glycosidase inhibitor inhibits the decomposition of disaccharides, it causes many side effects as follows. Namely, upon the intake of starch, the concentration of disaccharides in the intestines is increased so that diarrhea can occur due to an increase in osmotic pressure. Also, disaccharides are decomposed by intestinal bacteria, resulting in an increase in gas production.

It is known that green tea has a variety of important physiological effects, including antioxidative, antibacterial, anticancer, antihypertensive, and diabetic inhibitory effects, which are attributed to polyphenol extracted, isolated, and purified from the green tea.

However, the green tea is generally drunk after it draws in water at 60-80° C. for about 3-5 minutes, whereas a sufficient amount of polyphenol with high physiological activities will be extracted only if the green tea draws in boiling water over 30 minutes. Thus, the general drinking method of green tea has little effect to intake polyphenol at an amount sufficient to inhibit the increase of blood glucose.

Moreover, Korean patent laid-open publication No. 10-1999-11834 discloses a blood glucose-lowering agent containing a calcium compound.

### DISCLOSURE OF THE INVENTION

The present inventors have found that green tea polyphenol and calcium have a synergistic blood glucose-lowering effect by the function of inhibiting the stage of decomposing starch into disaccharides. On the basis of this finding, the present invention has been perfected.

The present invention provides a composition for lowering blood glucose, which contains a polyphenol extracted, isolated, and purified from green tea, and calcium.

The inventive composition for lowering blood glucose comprises 50.0-95.0% by weight of a polyphenol extracted, isolated, and purified from green tea, and 3.0-30.0% by weight of calcium.

Hereinafter, the present invention will be described in detail.

Green tea polyphenol powder used in the inventive composition for lowering blood glucose is prepared by extraction, isolation and purification according to the method described in Korean Patent No. 10-377313 (entitled “method for preparing green tea extract”). A preferred preparation method is as follows.

5-20 parts by weight of water is added to 1 part by weight of hot air-dried green tea leaf powder, and heated for 15 minutes to 2 hours. Then, the green tea leaf powder is removed, and the remaining extract is cooled to remove precipitates. The obtained extract is heated again and cooled to remove precipitates. The resulting extract is dried to obtain a green tea extract. The heating is preferably carried out at a temperature of 60-110° C., and the drying is preferably performed by a spray drying method. The green tea extract is preferably in the form of powder.

Calcium used in the inventive composition for lowering blood glucose comprises at least one selected from the group consisting of calcium carbonates (eggshell calcium, oyster shell calcium, seaweed calcium, and pearl calcium), calcium phosphate, calcium chloride, calcium lactate, calcium citrate, calcium gluconate, whey calcium, milk calcium, calcium pentothenate, peptide calcium, and chitosan calcium.

The inventive composition for lowering blood glucose shows the synergistic inhibition of blood glucose increase as compared to the case of the administration of green tea polyphenol or calcium alone. Thus, the inventive composition for lowering blood glucose will be useful as an agent for the prevention and treatment of diabetes, or health food, etc.

In addition to green tea polyphenol and calcium as active ingredients, the inventive composition for lowering blood glucose may contain other active ingredients which show blood glucose-lowering activities.

In addition to green tea polyphenol and calcium as active ingredients, the inventive composition may further contain other active ingredients which show blood glucose-lowering activity or other pharmacological effects.

For administration, the inventive composition may further contain at least one pharmaceutically or food-acceptable carrier, in addition to the active ingredients as described above. Examples of the pharmaceutically or food-acceptable carrier include saline solution, sterile water, Ringer's solution, buffered saline solution, dextrose solution, maltodextrin solution, glycerol, ethanol and a mixture of two or more thereof. If necessary, the inventive composition may also contain other conventional additives, such as antioxidants, buffers and bacteriostatic agents. Moreover, the inventive composition may additionally contain diluents, dispersants, surfactants, binders and lubricants in order to formulate it into injection formulations, such as aqueous solution, suspension and emulsion, pills, capsules, granules and tablets. Furthermore, the inventive composition may preferably be formulated depending on particular diseases and its components, using the method described in Remington's Pharmaceutical Science (latest edition), Mack Publishing Company, Easton Pa., which is a suitable method in the relevant field of art.

The inventive composition may be administered orally or parenterally. Preferably, the inventive composition is administered orally in the form of liquid, powder, capsule, tablet, syrup, and the like, but the administration form of the composition is not specifically limited. The dosage of the inventive composition can vary depending on various factors, including patient's weight, age, health condition, sex and diet, and administration time, administration route, secretion rate, disease severity, etc., it is preferable that the inventive composition be administered at a daily dosage of 5-100 mg/kg one time or several times.

For the prevention and treatment of diabetes, the inventive composition for lowering blood glucose may be used alone or in combination with surgical operations, radiation therapies, hormone therapies, chemical therapies, and other methods using biological reaction regulators.

A food formulation of the inventive composition is prepared by any conventional method. The inventive composition can be either dried together with a carrier and then encapsulated, or formulated into tablets, granules, powders, drinks, porridges, or all other forms.

### BEST MODE FOR CARRYING OUT THE INVENTION

The present invention will hereinafter be described in further detail by examples. It is to be understood, however, that these examples are presented to provide a better understanding of the present invention and not intended to limit the scope of the present invention.

### PREPARATION EXAMPLE 1

### Formulation Containing Green Tea Polyphenol and Calcium

A capsule formulation was prepared which contains 838 mg of green tea polyphenol powder, 70 mg of calcium chloride, 90 mg of vitamin C, 2 mg of iron supplement, and other excipients.

### COMPARATIVE EXAMPLE 1

### Calcium-Containing Formulation

A capsule formulation was prepared which contains 70 mg of calcium chloride, 90 mg of vitamin C, 2 mg of iron supplement, and other excipients. It was prepared in the same manner as in Preparation Example 1 except that the green tea polyphenol powder was not used.

### COMPARATIVE EXAMPLE 2

### Green Tea Polyphenol-Containing Formulation

A capsule formulation was prepared which contains 838 mg of green tea polyphenol powder, 90 mg of vitamin C, 2 mg of iron supplements, and other excipients. It was prepared in the same manner as in Preparation Example 1 except that the calcium chloride was not used.

### TEST EXAMPLE 1

### Effect of Inventive Composition on Digestive Absorption of Starch

In order to examine the effect of the inventive composition on the digestive absorption of starch, the measurement of blood glucose was performed as follows.

Splague-Dowley male white rats weighing 220-250 g were fasted for 4 hours, and divided into five groups consisting of a control group, a group administered with starch, a group administered with starch and calcium, a group administered with starch and green tea polyphenol, and a group administered with starch and green tea polyphenol-calcium, each group consisting of 7 animals. Specifically, the animal groups were administered with the following test samples:

Control group: 3 ml tap water;

Group administered with starch: 3 ml solution of 1 g starch in tap water;

Group administered with starch and calcium: 3 ml mixture of 1 g starch and 60 mg calcium formulation of Comparative Example 1 dissolved in tap water;

Group administered with starch and green tea polyphenol: 3 ml mixture of 1 g starch and 60 mg green tea polyphenol formulation of Comparative Example 2 dissolved in tap water; and

Group administered with starch and green tea polyphenol-calcium: 3 ml mixture of 1 g starch and 60 mg green tea polyphenol-calcium formulation of Preparation Example 1 dissolved in tap water.

The five test samples were administered orally to the white rats. Before the sample administration and 40 minutes after the sample administration, the tail of the white rats was incised from which blood was collected and measured for blood glucose level.

The measurement results are given in Table 1 below.

TABLE 1
Effect of inventive green tea polyphenol-calcium composition on blood
glucose of white rats administered with starch

| | Blood glucose level (mg/dl) | | |
|---|---|---|---|
| | | 40 minutes | Change |
| | Just before | after | in blood |
| | adminis- | adminis- | glucose |
| Test group | tration | tration | level |
| Control group | 97 ± 7 | 97 ± 8 | 0 |
| Group administered with starch | 96 ± 5 | 127 ± 9 | 31 |
| Group administered with | 96 ± 8 | 126 ± 8 | 30 |
| starch + calcium | | | |
| Group administered with starch + | 98 ± 6 | 119 ± 7 | 21 |
| green tea polyphenol | | | |
| Group administered with starch + | 97 ± 7 | 111 ± 5 | 14 |
| green tea polyphenol + calcium | | | |

Numerical value in Table 1: average value ± standard deviation

As shown in Table 1, the control group administered with tap water alone showed little or no change in blood glucose level at 40 minutes after administration as compared to that before its administration. The starch-administered group showed an increase of 31 mg/dl in blood glucose level at 40 minutes after starch administration as compared to that before starch administration. The group administered with starch and calcium showed an increase of 30 mg/dl in blood glucose level at 40 minutes after administration as compared to that before administration, indicating no difference from that of the group administered with starch alone. The group administered with starch and green tea polyphenol showed an increase of 21 mg/dl in blood glucose level at 40 minutes after administration as compared to that before administration, indicating that the increase in blood glucose level was about 32% inhibited as compared to that of the group administered with starch alone. The group administered with starch and green tea polyphenol-calcium showed an increase of 14 mg/dl in blood glucose level 40 minutes after administration as compared to that before administration, indicating that the increase in blood glucose level was about 54% inhibited as compared to that of the group administered with starch alone.

As a result, the inventive composition containing green tea polyphenol-calcium shows a greater inhibition of blood glucose increase than that of the composition containing green tea polyphenol or calcium alone. This suggests that the inventive composition shows a superior inhibitory effect against the increase of blood glucose by inhibiting the digestive absorption of starch.

### TEST EXAMPLE 2

### Blood Glucose-Lowering Effect of Green Tea Polyphenol-Calcium Composition in Diabetic White Rats

In order to examine the effect of the inventive composition on diabetic treatment, the following test was performed.

Splague-Dowley male white rats weighing 220-250 g were fasted for 12 hours. Then, a solution of alloxan in physiological saline was injected into the white rats at the amount of 80 mg/kg of body weight to induce diabetes in the rats. The diabetes-induced white rats were bred for 7 days, and then white rats with blood glucose levels of more than 350 mg/100 ml were selected for use in the test.

The diabetic white rats were fasted for 12 hours, and then divided into five groups consisting of a control group, a group administered with starch, a group administered with starch and calcium, a group administered with starch and green tea polyphenol, and a group administered with starch and green tea polyphenol-calcium, each group consisting of five animals. The conditions of the test samples were the same as in Test Example 1 above, and administered orally to the diabetic white rats. Before the sample administration and 40 minutes after the sample administration, the tail of the rats was incised from which blood was collected and measured for blood glucose level.

The measurement results are given in Table 2 below.

TABLE 2
Effect of inventive green tea polyphenol-calcium composition on blood
glucose level of diabetic white rats administered with starch

| | Blood glucose levels (mg/dl) | | |
|---|---|---|---|
| | | 40 minutes | Change |
| | Just before | after | in blood |
| | adminis- | adminis- | glucose |
| Test groups | tration | tration | level |
| Control group | 319 ± 21 | 312 ± 22 | −7 |
| Group administered with starch | 327 ± 25 | 406 ± 29 | 79 |
| Group administered with starch + | 324 ± 28 | 404 ± 31 | 80 |
| calcium | | | |
| Group administered with starch + | 326 ± 31 | 379 ± 27 | 53 |
| green tea polyphenol | | | |
| Group administered with starch + | 321 ± 27 | 352 ± 25 | 31 |
| green tea polyphenol + calcium | | | |

Numerical value = average value ± standard deviation

As shown in Table 2, the control group administered with tap water alone showed little or no change in blood glucose level at 40 minutes after administration as compared to that before administration. The group administered with starch showed an increase of 79 mg/dl in blood glucose level at 40 minutes after starch administration as compared to that before starch administration. The group administered with starch and calcium showed an increase of 80 mg/dl in blood glucose level at 40 minutes after administration as compared to that before administration, indicating no difference from that of the group administered with starch alone. The group administered with starch and green tea polyphenol showed an increase of 53 mg/dl in blood glucose level at 40 minutes after administration as compared to that before administration, indicating that an increase in blood glucose level was about 32% inhibited as compared to that of the group administered with starch alone. The group administered with starch and green tea polyphenol-calcium showed an increase of 31 mg/dl in blood glucose level 40 minutes after administration as compared to that before administration, indicating that an increase in blood glucose level was about 60% inhibited as compared to that of the group administered with starch alone.

As a result, the inventive composition containing green tea polyphenol-calcium shows a greater inhibition of blood glucose increase than that of the composition containing green tea polyphenol or calcium alone. This suggests that the inventive composition has a superior inhibitory effect on post-meal blood glucose increase in diabetes.

### TEST EXAMPLE 3

### Kidney and Liver Toxicity Tests on Inventive Composition Containing Green Tea Polyphenol and Calcium

In order to examine whether the inventive composition has toxicity or not, the inventive composition was administered to white rats on which liver and kidney function tests were then performed as follows.

Splague-Dowley male while rats weighing 220-250 g were fasted for 12 hours and then divided into two groups consisting of a control group and a group administered with the inventive green tea polyphenol-calcium composition, each group consisting of 10 animals.

The control group was administered orally with 2 ml of tap water, and the group administered with the inventive green tea polyphenol-calcium composition was administered orally with a solution of 200 mg of the green tea polyphenol-calcium composition of Preparation Example dissolved in 2 ml of tap water. This procedure was repeated three times at an interval of 3 days, and after 3 days, the white rats were fasted for 4 hours and then sacrificed. Blood was collected from the abdominal artery of the white rats, and measured for blood glucose level and the amounts of BUN, sGOT, sGPT, alkaline phosphatase and creatine, using biochemical blood analysis equipment.

The measurement results are given in Table 3 below.

TABLE 3
Examination of blood enzymes and chemical substances in white rats
administered with inventive green tea polyphenol-calcium composition

| | | Group administered with |
|---|---|---|
| | Control group | inventive composition |
| Glucose (mg/dl) | 98 ± 11 | 95 ± 8 |
| BUN (mg/dl) | 14.1 ± 1.5 | 13.1 ± 1.6 |
| Creatine (mg/ml) | 0.7 ± 0.1 | 0.7 ± 0.1 |
| sGOT (U/L) | 62 ± 9 | 61 ± 7 |
| sGPT (U/L) | 35 ± 6 | 36 ± 8 |
| Alkaline phosphatase (U/L) | 111 ± 17 | 106 ± 15 |

Numerical value = average value ± standard deviation

As shown in Table 3, there was no difference not only in the amounts of sGOT, sGPT and alkaline phosphatase, liver damage markers, but also in the amount of BUN and creatine, kidney damage markers, between the control group and the group administered with the inventive green tea polyphenol-calcium composition.

This suggests that the present composition containing green tea polyphenol and calcium does not cause toxicity in the liver or kidneys of rats.

### INDUSTRIAL APPLICABILITY

As apparent from the foregoing, the inventive composition for lowering blood glucose inhibits the digestive process of starch, thus significantly inhibiting the increase of blood glucose level. Thus, the inventive composition for lowering blood glucose can inhibit the increase of blood glucose level without reducing the intake of starch, suggesting that it will be useful for diabetic patients.

1. A composition for lowering blood glucose, which contains a polyphenol extracted, isolated, and purified from green tea, and calcium.

2. The composition of claim 1, which contains 50.0-95.0% by weight of the green tea polyphenol powder and 3.0-30.0% by weight of the calcium.

3. The composition of claim 1, wherein the calcium comprises at least one selected from the group consisting of calcium carbonates (eggshell calcium, oyster shell calcium, seaweed calcium, and pearl calcium), calcium phosphate, calcium chloride, calcium lactate, calcium citrate, calcium gluconate, whey calcium, milk calcium, calcium pentothenate, peptide calcium, and chitosan calcium.
